(19) United States
(12) Patent Application Publication
(10) Pub. No.: US 2009/0053337 A1
(43) Pub. Date: Feb. 26, 2009

(54) COMPOSITION AND METHOD OF IMPROVING SKIN BARRIER FUNCTION OF COMPROMISED SKIN

(75) Inventors: Isabelle HANSENNE, Westfield, NJ (US); Hani Fares, Somerset, NJ (US); Christian Oresajo, Nanuet, NY (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/842,193

(22) Filed: Aug. 21, 2007

(51) Int. Cl. A61K 36/15; A61Q 17/00

(52) U.S. Cl. 424/770

(57) ABSTRACT

A composition and method of improving skin barrier function of compromised skin by applying onto the compromised skin a composition containing: (a) a complex comprised of a metal-copper-antioxidant botanical pigment; (b) an extract of pinus pinaster bark; (c) an extract of pilewort; and (d) a carrier vehicle.

### BACKGROUND OF THE INVENTION

The present invention is directed to a composition and method of improving the skin barrier function of compromised skin. More particularly, by applying a composition containing a combination of: a chemically-bound copper complex; an extract of Pinus Pinaster Bark; and an extract of pilewort, the skin barrier function of compromised skin can be improved.

Skin is responsible for acting as a barrier in order to protect the body from physical injury, sense the environment, regulate body temperature, eliminate waste, and providing protection from microbial invasions. When the skin becomes damaged, its ability to perform these functions is compromised.

Skin has three layers: the epidermis, the dermis and the subcutaneous tissues. The epidermis is the top layer of the skin, and is responsible for providing a mechanical barrier to protect the body, generating new cells, protecting against solar radiation, and attracting and consuming antigens. The stratum corneum is the top layer of the epidermis and is where moisture content of the skin is regulated.

The stratum corneum is the “brick and mortar” of the skin. Keratin, a protein that provides structure to the skin, forms the “brick” component of the stratum corneum. The “mortar” between the bricks of keratin is comprised of phospholipid bilayers, commonly referred to as skin lipids. In healthy skin, the keratin and lipids join together to provide a barrier that keeps irritants out and the appropriate amount of moisture in.

When skin is compromised, it can no longer serve as an effective barrier, thus enabling external irritants and potentially pathogenic organisms to penetrate the stratum corneum into the lower layers of the epidermis. Moreover, moisture present in the skin is also allowed to travel to the surface of the skin where it evaporates. The result is dry, irritated skin which may harbor potentially pathogenic organisms.

### BRIEF SUMMARY OF THE INVENTION

The present invention is directed to a method of improving skin barrier function of compromised skin by applying onto the compromised skin a composition containing:

(a) a complex comprising metal-copper-antioxidant botanical pigment;
(b) an extract of pinus pinaster bark;
(c) an extract of pilewort; and
(d) a carrier vehicle.

The present invention is also directed to a composition for use in improving skin barrier function of compromised skin having:

(a) a complex comprising metal-copper-antioxidant botanical pigment;
(b) an extract of pinus pinaster bark;
(c) an extract of pilewort; and
(d) a carrier vehicle.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

It has been surprisingly discovered that by combining: a complex comprising metal-copper-antioxidant botanical pigment; an extract of pinus pinaster bark; and an extract of pilewort, a synergistic improvement of skin barrier function can be realized in compromised skin.

A complex comprising metal-copper-antioxidant botanical pigment is a compound capable of delivering cuprous copper ion (Cu2+) to copper-dependent enzymes in the skin. Examples thereof include, but are not limited to, sodium, zinc, iron, silver and magnesium.

Similarly, any cosmetically/dermatologically suitable botanical pigment may be bound to the copper ion without departing from the spirit of the invention. One example thereof is chlorophyllin.

The complex comprising metal-copper-antioxidant botanical pigment is typically present in the composition of the present invention in an amount of from about 0.0001 to about 5% by weight, preferably from about 0.001 to about 3% by weight, and more preferably from about 0.01 to about 1% by weight, based on the weight of the composition.

A preferred complex for use in the present invention is sodium-copper-chlorophyllin such as the one disclosed in US 2007/0148224, the entire content of which is hereby incorporated by reference.

The extract of pinus pinaster bark is typically present in the composition of the present invention in an amount of from about 0.01 to about 10% by weight, preferably from about 0.1 to about 5% by weight, and more preferably from about 0.2 to about 4% by weight, based on the weight of the composition.

Extract of pinus pinaster bark is commercially available from Horphag Research Group under the tradename Pycnogenol®.

The extract of pilewort, also known as ranunculus ficaria, is typically present in the composition of the present invention in an amount of from about 0.01 to about 10% by weight, preferably from about 0.1 to about 5% by weight, and more preferably from about 0.2 to about 4% by weight, based on the weight of the composition.

Extract of pilewort is commercially available from Gattefosse under the tradename Gatuline® A.

Suitable carrier vehicles for use in the present invention include, but are not limited to, propylene glycol, butylene glycol, pentylene glycol, isopentyl glycol, ethoxydiglycol, dimethyl isosorbide, acetamide MEA, tetrahydropiperine, and PEG glyceryl ethers.

The present invention will be better understood from the examples which follow, all of which are intended for illustrative purposes only and are not intended to limit the scope of the invention in any way.

### EXAMPLES

Inventive Composition

| Ingredient | % by weight | |
|---|---|---|
| Water | 81.70 | |
| Pentylene glycol | 4.00 | |
| Butylene glycol | 4.00 | |
| Denatured alcohol | 3.50 | |
| Pilewort extract | 1.50 | |
| Carbomer | 1.10 | |
| Pinus pinaster bark extract | 1.00 | |
| Sodium lactate | 1.60 | |
| Phenoxyethanol | 0.75 | |
| Sodium hydroxide | 0.75 | |
| Sodium-copper-chlorophyllin complex | 0.10 | |
| Total | 100% | by weight |

Comparative Composition 1

| Ingredient | % by weight | |
|---|---|---|
| Water | 84.20 | |
| Pentylene glycol | 4.00 | |
| Butylene glycol | 4.00 | |
| Denatured alcohol | 3.50 | |
| Carbomer | 1.10 | |
| Sodium lactate | 1.60 | |
| Phenoxyethanol | 0.75 | |
| Sodium hydroxide | 0.75 | |
| Sodium-copper-chlorophyllin | 0.10 | |
| complex | | |
| Total | 100% | by weight |

Comparative Composition 2

| Ingredient | % by weight | |
|---|---|---|
| Water | 81.80 | |
| Pentylene glycol | 4.00 | |
| Butylene glycol | 4.00 | |
| Denatured alcohol | 3.50 | |
| Pilewort extract | 1.50 | |
| Carbomer | 1.10 | |
| Pinus pinaster bark extract | 1.00 | |
| Sodium lactate | 1.60 | |
| Phenoxyethanol | 0.75 | |
| Sodium hydroxide | 0.75 | |
| Total | 100% | by weight |

A clinical study was performed on 3 groups of subjects, each group having 10 subjects therein, for a total of 30 subjects. All of the subjects suffered from a form of compromised skin in order to determine what, if any, effect the compositions would have on trans-epidermal water loss (TEWL). The study occurred over a 4 week period with 3 measurements being taken, an initial measurement on day 1 to obtain a baseline, followed by measurements at 2 and 4 weeks thereafter

Each group of subjects was given one of the above-identified compositions to apply onto the affected area of their skin. Twice a day, each and every day, the affected area was to be washed with a cleanser, and then treated with the composition, followed by application of a sunscreen.

TEWL was measured with a probe conventionally used to determine TEWL. Statistical significant is realized when a “p” value of 0.05 or less is observed.

The results of the study are found in the Table below.

| | Inventive | Comparative | Comparative |
|---|---|---|---|
| | Composition | Composition 1 | Composition 2 |
| Week 2 | p = 0.015 | 0.110 | 0.733 |
| Week 4 | p = 0.003 | 0.007 | 0.915 |

As can be seen from the above data, trans-epidermal water loss experienced by compromised skin was reduced to a statistically significant level after only 2 weeks using the inventive composition. Comparative composition 1 did not exhibit statistically significant TEWL until week 4, while comparative composition 2 did not exhibit any at all.

What is claimed is:

1. A method of improving skin barrier function of compromised skin by applying onto the compromised skin a composition containing:
(a) a complex comprised of a metal-copper-antioxidant botanical pigment;
(b) an extract of pinus pinaster bark;
(c) an extract of pilewort; and
(d) a carrier vehicle.

2. The method of claim 1 wherein (a) is a sodium-copper-chlorophyllin complex.

3. The method of claim 1 wherein (a) is present in the composition in an amount of from about 0.0001 to about 5% by weight, based on the weight of the composition.

4. The method of claim 1 wherein (a) is present in the composition in an amount of from about 0.01 to about 1% by weight, based on the weight of the composition.

5. The method of claim 1 wherein (b) is present in the composition in an amount of from about 0.01 to about 10% by weight, based on the weight of the composition.

6. The method of claim 1 wherein (b) is present in the composition in an amount of from about 0.2 to about 4% by weight, based on the weight of the composition.

7. The method of claim 1 wherein (c) is present in the composition in an amount of from about 0.01 to about 10% by weight, based on the weight of the composition.

8. The method of claim 1 wherein (c) is present in the composition in an amount of from about 0.2 to about 4% by weight, based on the weight of the composition.

9. The method of claim 1 wherein (a) is present in an amount of from about 0.0001 to about 5% by weight, (b) is present in an amount of from about 0.01 to about 10% by weight, and (c) is present in an amount of from about 0.01 to about 10% by weight, all weights being based on the weight of the composition.

10. The method of claim 1 wherein (a) is present in an amount of from about 0.01 to about 1% by weight, (b) is present in an amount of from about 0.2 to about 4% by weight, and (c) is present in an amount of from about 0.2 to about 4% by weight, all weights being based on the weight of the composition.

11. A composition comprising:
(a) a complex comprised of a metal-copper-antioxidant botanical pigment;
(b) an extract of pinus pinaster bark;
(c) an extract of pilewort; and
(d) a carrier vehicle.

12. The composition of claim 11 wherein (a) is a sodium-copper-chlorophyllin complex.

13. The composition of claim 11 wherein (a) is present in the composition in an amount of from about 0.0001 to about 5% by weight, based on the weight of the composition.

14. The composition of claim 11 wherein (a) is present in the composition in an amount of from about 0.01 to about 1% by weight, based on the weight of the composition.

15. The composition of claim 11 wherein (b) is present in the composition in an amount of from about 0.01 to about 10% by weight, based on the weight of the composition.

16. The composition of claim 11 wherein (b) is present in the composition in an amount of from about 0.2 to about 4% by weight, based on the weight of the composition.

17. The composition of claim 11 wherein (c) is present in the composition in an amount of from about 0.01 to about 10% by weight, based on the weight of the composition.

18. The composition of claim 11 wherein (c) is present in the composition in an amount of from about 0.2 to about 4% by weight, based on the weight of the composition.

19. The composition of claim 11 wherein (a) is present in an amount of from about 0.0001 to about 5% by weight, (b) is present in an amount of from about 0.01 to about 10% by weight, and (c) is present in an amount of from about 0.01 to about 10% by weight, all weights being based on the weight of the composition.

20. The composition of claim 11 wherein (a) is present in an amount of from about 0.01 to about 1% by weight, (b) is present in an amount of from about 0.2 to about 4% by weight, and (c) is present in an amount of from about 0.2 to about 4% by weight, all weights being based on the weight of the composition.
